(19) United States
(12) Patent Application Publication
(10) Pub. No.: US 2012/0107368 A1
(43) Pub. Date: May 3, 2012

(54) COMPRESSIBLE TABLET MATERIAL HAVING AN OIL-CONTAINING ACTIVE SUBSTANCE, TABLET AS WELL AS METHOD AND DEVICE FOR THE PRODUCTION THEREOF

(75) Inventors: Edmont Stoyanov, Aalen (DE); Reinhard Vollner, Windesheim (DE); Tobias Götz, Rosenburg (DE)

(73) Assignee: J. RETTENMAIER & SOHNE GMBH & CO. KG, Rosenberg (DE)

(21) Appl. No.: 13/255,667

(22) PCT Filed: Mar. 15, 2010
(86) PCT No.: PCT/EP2010/001615

§ 371 Date: Dec. 21, 2011

(30) Foreign Application Priority Data
Mar. 13, 2009 (DE) 10 2009 012 788.7

(51) Int. Cl. A61K 9/14; A62C 13/62; A61K 47/38

(52) U.S. Cl. 424/400; 514/770; 239/304

(57) ABSTRACT

The invention relates to a compressible tablet material comprising the following components:
an oil containing an active substance;
silicified microcrystalline cellulose or microcrystalline cellulose as excipient.

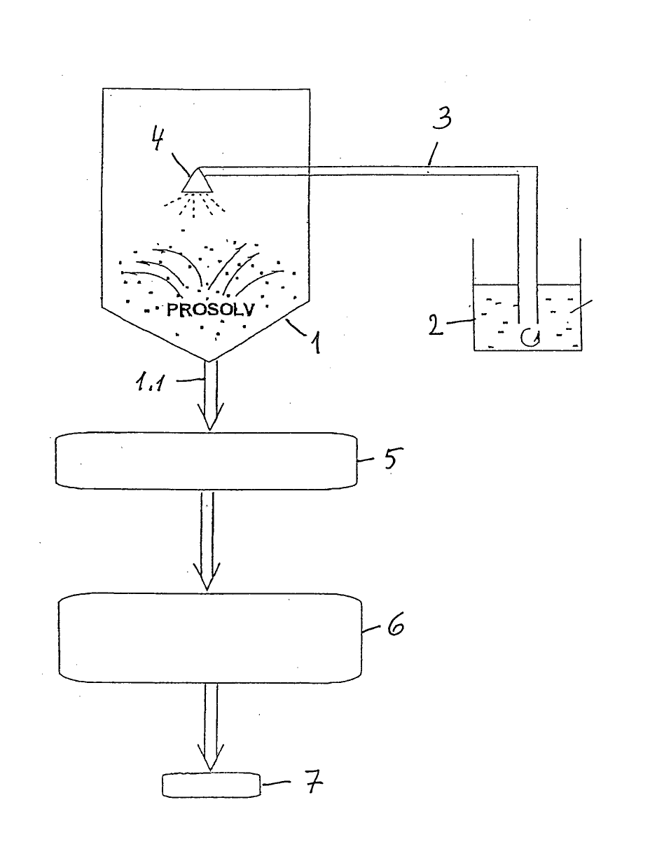

The invention relates to the subject matters specified in the title.

There are numerous active substances which are present as liquid oils. Examples of oily active substances having physiological action are

unsaturated fatty acids: ω-3 and ω-6 fatty acids (DHA)

poly-unsaturated alcohols: lutein, carotenes (vitamin A)

lipophilic phenols and phosphatidylcholines: tocopherols, lecithins

plant extracts having a high fat content: evening

primrose oil

solution of lipophilic active substances in oil

Each of the said groups has special chemical or pharmacological properties.

The active-substance-containing oil can be administered in liquid form. Capsules which hold the oil, primarily gelatine capsules come into consideration here. Such capsules are advantageous since they can be taken easily and slide through the oesophagus without any difficulties. In addition, the active substance in the gelatine shell is protected from light and oxygen.

A disadvantage of capsules is the high manufacturing costs. Consequently endeavours are being made to transfer the oily substance into tablet form. For this purpose powdery substances are added to the active-substance-containing oil which are intended to absorb the oil. After adding the powdery substance the mixture is pressed into tablets. However, the tablets are of low strength and therefore break very easily. The uniformity of the individual tablets is also unsatisfactory. Finally, it takes a relatively long time before the tablet breaks down in the stomach after it has been taken. Also the absorption of oil in the powder is not sustainable; over longer periods of time, unmixing of oil and powder take place.

It is the object of the invention to provide means or steps by which means a compressible tablet material having an oily active substance can be manufactured and a tablet which can be manufactured from this, which has the requisite properties, i.e. a high content of oily active substance which has the necessary strength, and all the other properties required of a tablet.

This object is achieved by the features of the independent claims.

The inventors have accordingly identified that an ideal excipient for producing a compressible tablet material or for manufacturing a tablet is one of the following substances:

SMCC (silicified microcrystalline cellulose)

MCC (microcrystalline cellulose)

The said excipients are not only highly absorptive in relation to oil. They are also suitable for compressing into a tablet after mixing with the oily active substance.

At least one of the two said excipients, therefore SMCC and/or MCC, should have a particle size of 5 μm to 500 μm, preferably 50-500 μm.

The inventors have further identified that certain process steps are advantageous in order to manufacture a compressible tablet material according to the invention. The following process steps are therefore expedient:

(a) The oily active substance is converted into an emulsion, for example, by adding water. The emulsification can take place at room temperature but also at temperatures of 30, 40 . . . -100° C.

(b) The emulsion is then sprayed onto SMCC or MCC.

The oily active substance can also be brought together with SMCC or MCC in a fluidized bed.

1) Experiments

Active substance: all-rac-α-tocopherol acetate (DL-α-tocopherol acetate), manufacturer: Audor Pharma GmbH, batch AP UT06080183 T

Properties: viscous oil

sensitive to oxygen

thermostable

2) Additives

PROSOLV SMCC®90—JRS Pharma

Sodium dodecyl sulphate—MERCK

EMDEX-JRS Pharma

PROSOLV is a silicified microcrystalline cellulose having a particle size of 110 μm and a silicic acid content of 2-10%.

EMDEX is a mixture of glucose and maltodextrin.

3) Technical Equipment

Fluidized bed granulator with “top spray” and two-material nozzle—Diosna Minilab

Turbula Mixer—Ultra-TURRAX, IKA

Scales—Ohaus

Tablet press—Korsch KO

4) Method

200 g of PROSOLV SMCC 90 was placed in a fluidized bed and heated to 45° C. Then 1 g of sodium dodecyl sulphate was dissolved in 25 ml of water at room temperature and 40 g of tocopherol acetate was added slowly whilst stirring vigorously. The emulsion thus obtained was rapidly sprayed onto PROSOLV SMCC 90 and dried in the fluidized bed.

5) Results

5.1 The Co-Processing Powder (Granular Material)

This comprises the tablet material to be compressed.

The experimental result is illustrated in the following Table 1.

TABLE 1

| Parameter | |
|---|---|
| Oil content | 15.80% |
| Residual moisture | 4.6-4.9% |
| Pourability (FlowDex) | 3 g/s, 32 mm |
| Angle of repose | 53-54° |
| Bulk weight | 297/305 g/l |

The powder thus produced creates problems during pressing since the angle of repose is too high. In order to improve the pourability, approx. 50% EMDEX was added as another excipient.

Table 1 shows that satisfactory results were achieved with regard to the remaining most important parameters. In particular the oil content of the granular material can be regarded as acceptable. However, the angle of repose and therefore the pourability are in need of improvement.

5.2 Possibilities for Improvement

An attempt was made to increase the oil content and/or compressibility. In so doing, the following facts have resulted:

The choice of the particle size of the excipient can be important.

The best particle size can be determined by matching active substance and excipient. An empirical determination from case to case is thereby easily possible. Suitable particle sizes lie between 50 and 500 μm. The following ranges have proved to be suitable:

50, 60, 70, 80 . . . 500 μm.

A pairing of active substance and excipient can therefore be found in which, for example, a range of 130-160μm is optimal.

The proportion of silicic acid (SiO₂) is particularly important. In general, a higher silicic acid content is favourable. Values between 2 and 30% were used in the experiments. Each individual percentage between these limiting values can be suitable, for example, 14, 15, 16% or 21, 22, 23%.

It has also been found that certain excipients—in addition to the additives SMCC or MCC, can be helpful with regard to an improvement of the oil absorbing capacity and/or compressibility. Sugar or sugar alcohols are considered as further excipients, therefore for example
Glucose
Lactose
Saccharose
Sorbitol
Mannitol
Xylitol.

A mixture of glucose and maltodextrin has proved particularly suitable.

6. Examples of Tablets Obtained from Experiments

TABLE 2
Important tablet parameters

| Parameter | Desired | Actual |
|---|---|---|
| Oil content | 8-10% | 8% |
| Tablet weight | 600 mg | 600 mg |
| Tablet hardness | min. 6 kp | 5.9-6.3 kp |
| Disintegration | max. 20 min | 26-44 sec. |
| Weight uniformity | max. 5% | 2-4% |
| Product uniformity | 0.5-2.0% | 4.8% |
| Abrasion | <1% | 0.11% |

The planned tablet hardness of 6 kp and abrasion of less than 1% were achieved. The disintegration times were even significantly better than we had expected (Table 2). This is probably because almost 50% of the tablet consists of EMDEX. In addition, it is also important that the PROSOLV particles are coated with an oil that prevents contact between the individual particles during compressing. Thus, no stable matrix can be formed, that is, the tablet is not too hard and disintegrates faster. 5% of a hardener (Kollidon V64 fine) was added to the mixture to increase the tablet hardness (Table 3).

TABLE 3
Kollidon formulation

| # | mg | % |
|---|---|---|
| Granular material | 315.0 | 52.5 |
| EMDEX | 255.0 | 42.5 |
| Kollidon V64 fine | 30.0 | 5.0 |
| Total | 600.0 | 100.0 |

The tablet hardness has thus increased to 14-16 kp without influencing the disintegration and the abrasion. At room temperature (20-25° C.), the Kollidon tablets are stable and retain their good hardness. At 50° C. and 168 hours (1 week) in the dry cupboard, the Kollidon tablets also lose hardness. The addition of Kollidon has significantly increased the tablet hardness without any negative effects. The attainment of specific tablet parameters such as hardness, abrasion and disintegration has not been problematical in this case.

An installation for producing a compressible tablet material according to the invention is explained in detail with reference to the drawing. The following is shown in detail therein:

Container 1 contains an aqueous suspension of SMCC and specifically as a fluidized bed.

Container 2 contains an aqueous emulsion of vitamin E which forms the active substance. An agitator 2.1 is only shown schematically.

A pipeline 3 conveys the emulsion from container 2 to a spray nozzle 4. This sprays the active substance emulsion onto the additive suspension or into this depending on the arrangement of the spray nozzle.

The granular material is released from container I through its outlet 1.1 at a given time and enters into a container 5.

It is then transferred into another container 6. Additional excipients are added before or in the container 6, for example, a mixture of glucose and maltodextrin, as well as possibly a hardener. These two components are mixed together with the granular material in the container 6, for example, during a time period of half to one hour.

The mixture is then released from container 6 and supplied to a tabletting press which produces tablets 7.

The advantages of the invention can be summarized as follows:

The invention enables an oil containing an active substance (an oily active substance) to be tabletted.

Pre-granulation in a fluidized bed is expedient; in particular the active substance is protected, that is it does not or barely loses its properties.

The tablet hardness can be improved by excipients.

The manufacturing costs of the tablets are low, in any case substantially lower than the manufacturing costs of gelatin capsules.

### REFERENCE LIST

1 Container for additive
1.1 Outlet
2 Container for oily active substance
2.1 Agitator
3 Pipeline
4 Nozzle
5 Granular material container
6 Mixer
7 Tablet

1-14. (canceled)

15. Compressible tablet material having physiological effect, comprising the following components:
an oily active substance or an oil containing an active substance;
silicified microcrystalline cellulose (SMCC) as excipient; having a particle size of 5-500 μm.

16. The tablet material according to claim 15, characterised in that the silicified microcrystalline cellulose (SMCC) has a particle size of 50-250 μm.

17. The tablet material according to claim 15, characterised in that a silicic acid concentration lies between 5 and 20%.

18. A method for manufacturing a compressible tablet material having an oily active substance or an oil that contains an active substance, characterized by the following process steps:
an aqueous emulsion is formed from the oily active substance or the oil;
an aqueous suspension is formed from an excipient comprising silicified microcrystalline cellulose (SMCC) having a particle size of 5-500 μm and a silicic acid concentration between 1 and 30%;
the suspension is presented as a fluidized bed and the emulsion is sprayed onto the suspension.

19. The method according to claim 18, characterised in that the emulsion containing the active substance is sprayed onto or into the suspension containing the excipient.

20. The method according to claim 18, characterised in that another excipient is added to the mixture of active substance and excipient to improve the tablettability.

21. The method according to claim 20, characterised in that the additional excipient is a sugar or an alcohol or a mixture of these two.

22. The method according to claim 21, characterised in that the additional excipient contains at least one of the following substances:
glucose
lactose
saccharose
sorbitol
mannitol
xylitol.

23. An apparatus for manufacturing a compressible tablet comprising the following components:
an oily active substance or an oil that contains an active substance;
a silicified microcrystalline cellulose (SMCC) having a particle size of 5-500 μm and a silicic acid concentration between 1 and 30% as excipient,
comprising the following components:
a first container for receiving the excipient and for forming a fluidized bed from this;
a second container for receiving the oily active substance or the oil,
a conducting connection for transferring the oily active substance or the oil in emulsified form and for spraying onto the fluidized bed in the first container through a spray nozzle.

24. The apparatus according to claim 23, characterised in that a mixer is connected downstream of the first container.

25. The apparatus according to claim 23, characterised in that a device for supplying an additional excipient is connected downstream of the first container.

26. A tablet manufactured from a material according to claim 15 or by means of a method according to claim 18 by means of an apparatus according to claim 23.
